(19) United States
(12) Patent Application Publication
(10) Pub. No.: US 2016/0213782 A1
(43) Pub. Date: Jul. 28, 2016

(54) POLYURETHANES AS ORAL DRUG DELIVERY PLATFORM

(71) Applicant: Universiteit Gent, Gent (BE)

(72) Inventors: Jean Paul Remon, Melle (BE); Chris Vervaet, Kachtem (BE); Bart Claeys, Oostduinkerke (BE)

(21) Appl. No.: 15/084,734

(22) Filed: Mar. 30, 2016

Related U.S. Application Data
(63) Continuation-in-part of application No. PCT/EP2014/070780, filed on Sep. 29, 2014.

(30) Foreign Application Priority Data
Sep. 30, 2013 (EP) 13186662.6

(51) Int. Cl. A61K 47/34; A61K 31/522; A61K 31/167; B29C 45/00; A61K 9/00; A61K 9/20; B29C 45/18; B29C 45/47; A61K 31/138; A61K 31/155

(52) U.S. Cl. CPC A61K 47/34; A61K 31/138; A61K 31/522; A61K 31/167; A61K 31/155; A61K 9/0053; A61K 9/2031; A61K 9/2095; B29C 45/18; B29C 45/47; B29C 45/0001; B29K 2075/00

(57) ABSTRACT

A pharmaceutical composition for oral use including a release formulation formed by extrusion, said formulation in particular including a polyurethane polymer and an active agent. A process of manufacturing the formulation, uses and methods of treatment.

### CROSS-REFERENCE TO RELATED APPLICATIONS

This application is filed under 35 U.S.C. §111 as a continuation-in-part of International Patent Application No. PCT/EP2014/070780, filed on Sep. 29, 2014, which designates the United States and claims priority to European Application No. 13186662.6, filed on Sep. 30, 2013, the contents of which are hereby incorporated by reference in their entirety.

### FIELD OF THE INVENTION

The present invention in general relates to a pharmaceutical composition for oral use comprising a release formulation formed by extrusion, said formulation in particular comprising a polyurethane polymer and an active agent. The invention further includes a process of manufacturing the formulation, uses and methods of treatment.

### BACKGROUND TO THE INVENTION

Conventional pharmaceutical dosage forms are often associated with undesired drug level oscillations. For many drugs, however, controlled release may be desired, making conventional dosage forms less suitable. Therefore, controlled and/or sustained-release delivery systems are continuously being developed for multiple types of drugs. In said delivery systems, preferably drug level oscillations should be minimized and more constant controlled drug levels over time should be achieved. Even though various drug delivery systems are used for maximizing therapeutic index and reducing the side effects of the drug, oral route remains the preferred, promising and effective route for the administration of therapeutic agents. Low cost of therapy, ease of administration, flexibility in formulation and handling leads to higher level of patient compliance.

Ideally, controlled and/or sustained-release formulations should be capable of providing a therapeutically effective drug level which allows the practitioner to target the therapeutic window of drug efficacy, while controlling the drug levels. In addition, controlled and/or sustained-release formulations may also minimize the frequency of dosing, which has a positive impact on patient compliance. Hence, to be a useful drug carrier, a polymer needs to possess certain features. The polymeric carrier must contain an effective dose of the active agent and the rate of drug release from the carrier must occur at an acceptable rate.

Typical polymers used for sustained release formulations are ethylcellulose (EC), hydroxypropyl methyl cellulose (HPMC), ethylene vinyl acetate (EVA), polyvinyl acetate (PVA), poly lactic (co-glycolic) acid (PL(G)A), polycaprolactone (PCL), the methacrylate copolymers (Eudragit® RS/RL) and even fatty acids. However, despite the usefulness of several polymers, the drug load in these formulation is often limited (e.g. 30 wt. % or even less).

It is well known that a higher, good water soluble, drug load induces a (too) enhanced release profile via the creation of more pores in the micro-capillary network of the matrix. In addition, the use of swellable or disintegrating polymeric materials modifies the available surface area of the formulation, resulting in a (too) enhanced drug release profile, often with burst release at the start. Hence, problems typically encountered for matrix systems for the oral controlled release delivery of freely water-soluble drugs are dose dumping, the burst phenomenon, and difficulty in achieving 24-hour linear release profile.

In addition, currently available drug release formulations are often pellets or particles comprising a core and a coat, wherein the coating is applied in a batch manufacturing process. However, batch process manufacturing has many drawbacks. Furthermore, quality is assessed through sampling during the process, and if quality standards are not met, the entire batch is rejected. It is therefore desirable to manufacture pharmaceutical compositions by a continuous manufacturing method.

To this end, the design and synthesis of innovative polymeric materials with improved characteristics is continuously under investigation.

Polyurethanes form a class of thermoplastic elastomers and their use for biomedical applications is known and reviewed e.g. by Batyrbekov and Iskakov, 2012 and by Cherng J Y et al., 2013. Many biomedical devices are made from polyurethanes such as catheters, vascular grafts, cardiac valves, stents, mammary prostheses, ocular implants, and intravaginal rings (as disclosed in e.g. WO2008/089488, WO2009/094573, WO2012/134540, WO2010/120389 and by Clark et al., 2012). Polyurethane microcapsules as local drug delivery systems have also been studied (Hong and Park, 1999). However, the use of polyurethanes for oral applications is very limited and e.g. described by Subhaga C S et al., 1995, which however faces the burst phenomenon and requires the use of a solvent in the production process (film casting).

It was therefore an object of this invention to provide an extruded pharmaceutical formulation comprising a polyurethane and an active agent which overcomes the difficulties as specified above. It was demonstrated for the first time that thermoplastic polyurethanes (TPUR) enable the production of high dosage formulations with sustained release profiles. Unexpectedly the present inventors found that polyurethanes can be used to formulate oral drug delivery systems and that the amount of polymer to be used is very low in comparison to other sustained release formulations.

### SUMMARY OF THE INVENTION

The current invention provides a pharmaceutical formulation useful for oral and controlled delivery of an active ingredient. The present invention relates to a solid pharmaceutical dosage form for oral use, comprising an extrusion formed release formulation.

More specific, the present invention provides a controlled release formulation for oral use comprising a polyurethane and an active agent, and characterized in that said composition is obtainable by extrusion, in particular melt extrusion. In particular, the release formulation is a single-layered release formulation.

The dosage form may further comprise at least one pore forming, solubilizing and/or wetting agent.

This invention further provides the solid dosage form according to this invention for use as a medicament, in particular for the sustained or controlled release of one or more active agents. More particular, the invention relates to the use of a dosage form for the manufacture of a composition suitable for oral administration and for controlled release of an active agent.

Furthermore, the invention provides a solid dosage form for use in the treatment of a disease by delivering one or more active agents to a subject in controlled manner.

In a further aspect, the present invention provides a method for the controlled release of an active agent, said method comprising orally administering a solid pharmaceutical dosage form according to this invention.

In yet a further aspect, the present invention provides a method of preparing a release formulation for oral use, said method comprising providing and extruding a polyurethane and an active agent. Optionally the method further comprises the step of injection molding or casting.

The current invention also provides a dosage form obtainable by a process as defined herein.

### BRIEF DESCRIPTION OF THE DRAWINGS

FIG. 1: Mean dissolution profiles (±S.D.) of drug release from Pearlbond matrices in function of drug solubility: 65 wt. % TH (10 mg/ml, Δ), Dyph (330 mg/ml, □) and MPT (>1000 mg/ml, ×).

FIG. 2: Mean dissolution profiles (±S.D.) of drug release from Pearlbond matrices in function of drug load: 60% () and 65% MPT (×).

FIG. 3: Mean dissolution profiles (±S.D.) of drug release from Pearlbond matrices in function of PEG4000 concentration: Dyph/PU 65/35 (□) with 2 (▴), 5 (▾) and 10% (♦) of PEG4000, respectively.

FIG. 4: Mean dissolution profiles (±S.D.) of drug release from Pearlbond matrices in function of Tween80 concentration: Dyph/PU 65/35 (□) with 2 (▴), 5 (▾) and 10% (♦) of Tween80, respectively.

FIG. 5: Mean dissolution profiles (±S.D.) of drug release from Tecoflex matrix: Dyph/T80A 65/35 with 7.5 (dotted line) and 10% PEG 4000 (full line), respectively.

FIG. 6: Mean MPT plasma concentration (±S.D.) after oral administration of 200 mg MPT to dogs in the form of tablets containing MPT/Pearlbond 65/35 (▪) and as Slow-Lopresor 200 Divitabs® ().

FIG. 7A: In vitro release kinetics (mean±SD, n=3) of formulations containing acetominophen (Aceta) and different TPU grades (SP60D60, SP93A100 and TG2000).

FIG. 7B: In vitro release kinetics (mean±SD, n=3) of formulations containing diprophylline (Dyph, 7-(2,3-dihydroxypropyl)-theophylline) and different TPU grades (SP60D60, SP93A100 and TG2000).

FIG. 7C: In vitro release kinetics (mean±SD, n=3) of formulations containing Theophylline (Theo) and different TPU grades (SP60D60, SP93A100 and TG2000).

FIG. 8A: Mean serum concentration profiles (±S.D.) after oral administration to Beagle dogs (n=6) of injection-molded TPU-based tablet (60% (w/w) metformin.HCl 240 mg).

FIG. 8B: Mean serum concentration profiles (±S.D.) after oral administration to Beagle dogs (n=6) of injection-molded Glucophage® SR (½ tablet).

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. As used in the specification and the appended claims, the singular forms “a”, “an”, and “the” include plural referents unless the context clearly dictates otherwise. By way of example, “a compound” means one compound or more than one compound. Throughout the description and claims of this specification the word “comprise” and other forms of the word, such as “comprising” and “comprises,” means including but not limited to, and is not intended to exclude, for example, other additives, components, integers, or steps. The terms described above and others used in the specification are well understood to those in the art.

The current invention provides a pharmaceutical formulation useful for oral and controlled delivery of one or more active agents. The formulation is a solid dosage form for delivering therapeutics in a controlled fashion over a prolonged period of time, maintaining the desired therapeutic level. A “solid” dosage form refers to a dosage form of definite shape and volume, not liquid or gaseous. As used herein, “controlled” release (optionally also referred to as sustained release) refers to the release of an active agent from a dosage form at a predetermined rate. Controlled release implies that the active agent is not released in an unpredictable fashion and that the majority of the active ingredient does not “burst” off of the dosage form upon contact with a biological environment. The drug levels are maintained within the therapeutic window to avoid potentially hazardous peaks in drug concentration following ingestion and therapeutic efficiency is maximized. In a particular embodiment, the active agent in the dosage form as described herein is gradually released for at least about 80%, 85%, 90%, 95% or 100% within 24 hours.

The present invention relates to an oral dosage form, comprising an extrusion formed release formulation, comprising:
a polyurethane; and
an active agent.

In a further embodiment, the invention relates to a dosage form for use in oral delivery of an active agent, wherein said dosage form comprises a melt extrusion formed release formulation.

Next to the use in a single-layered release formulation, the polyurethanes can optionally be used in multi-layered release formulations capable of releasing two or more different types of drug in a different manner. The present invention thus also relates to a multi-layered oral dosage form comprising a co-extrusion formed release formulation, comprising a core and one or more coat layers, wherein one or more of these layers, and preferably all, comprise a polyurethane; and wherein one or more of said layers comprises an active agent. In a particular embodiment however, the release formulation of the present invention does not comprise a surface coating or a coating layer, e.g. based on a non-polyurethane polymer. More particular, the release formulation does not comprise a drug impermeable coating (e.g. for preventing leakage of the active agent) or a coating intended to prevent a burst effect. The impermeability may be determined by standard methods, such e.g. as described herein, performing dissolution tests in the release formulation and analyzing which coating thickness or material does not allow the release of the active agent from the release formulation during the desired duration of controlled release.

The polyurethane used in the present invention typically is a thermoplastic polyurethane (TPUR). TPURs are formed by the reaction of diisocyanates with diols. The diisocyanate (NCO-R-NCO) can comprise an aliphatic or aromatic function, while the diol function can comprise a polyester, a polyether, a polysiloxane, a polycaprolactone, a polycarbonate, etc. In particular, the polyurethane is an aliphatic or aromatic polyurethane. In a further embodiment the polyurethane is a polyether, a polyester or a polycaprolactone based polyurethane, or a combination thereof. Furthermore, the polyurethane can be either hydrophobic, hydrophilic or a combination thereof. TPURs are available in various molecular weights, different types of soft segments (SS) with different lengths and variable SS/HS (hard segment) ratios. They can be classified as hydrophilic or hydrophobic, depending on the soft segment in the polymer. The soft segment determines the characteristics of the polyurethane, for example, Tecoflex™ is hydrophobic because of its polybutyleenoxide (PBO) soft segment, whereas Tecophilic™ is hydrophilic due to its polyethylene glycol (PEG) soft segment. Both polymers have the same HS composition: HMDI as isocyanate and 1,4-butanediol. Hydrophobic and hydrophilic TPU derivatives exist which both have been demonstrated herein as successful in the production of sustained release formulations.

The polyurethane can be a medical grade or non-medical grade polyurethane. A variety of medical grade polyurethanes may be used. For example, in some embodiments, the polyurethanes are the reaction product of a polymeric diol, a short chain diol, and a diisocyanate. Diisocyanates include, but are not limited to, symmetrical molecules like methylene-bis-cyclohexyl diisocyanates, 1,4 cyclohexyl diisocyanate, dicyclohexyl methane diisocyanate (HMDI) and hexamethylene diisocyanate. Short chain diols include, but are not limited to 1,4 butane diol or similar symmetrical diols or assymetrical diols like 1,2 propane diol. Polymeric diols include, but are not limited to, poly tetra methylene ether glycol (PTMEG) chosen from a molecular weight of 500 to 10,000. In some embodiments, the polyurethane comprises the reaction product of dicyclohexyl methane diisocyanate, a PTMEG having a molecular weight of between about 500 and about 10,000, and 1,4 butane diol. In other embodiments, the PTMEG has a molecular weight of about 1,000 to about 2,000. In some embodiments, the number of moles of dicyclohexyl methane diisocyanate is equal to the sum of the number of moles of PTMEG and the number of moles of 1,4 butane diol and the molar ratio of 1,4 butane diol to PTMEG is between about 1 to 1 and about 1.5 to 0.5. In some embodiments, the polyurethane has an average molecular weight of about 120,000 to about 180,000 and a weight average molecular weight of about 285,000 to about 335,000. Suitable polyurethanes and their synthesis are described in detail in U.S. Pat. No. 4,523,005 and US20110112270, which is hereby incorporated by reference in its entirety. In addition, thermoplastic polyurethanes are commercially available and provided by e.g. Lubrizol, DSM, Bayer, etc. Tecophilic™ is an example of a hydrophilic and aliphatic TPU (e.g. Tecophilic™ SP60D60, SP93A100 and TG2000; Merquinsa—a Lubrizol Company, Ohio, USA). Tecoflex™ is an example of a hydrophobic TPU (e.g. Tecoflex™ EG72D; Merquinsa—a Lubrizol Company, Ohio, USA) The hard segment of Tecophilic™ has the same chemical composition as the hard segment of Tecoflex™ EG72D.

Any of the polyurethanes described above may be used alone or in combination. In general, the dosage form may comprise about and between 10-90 wt. %, 10-80 wt. %, or 10-70 wt. % of a polyurethane polymer, in particular about and between 10-60 wt. % of a polyurethane polymer, more in particular about and between 10-50 wt. %, and even more in particular about and between 20-50 wt. % of a polyurethane polymer, including 20-45 wt. %, 20-40 wt. %, 20-35 wt. % and 20-30 wt. %. Hence, the oral dosage form of the present invention may comprise at least or up to about 10, 11, 12, 13, 14, 15, 16, 17, 18, 19, 20, 21, 22, 23, 24, 25, 26, 27, 28, 29, 30, 35, 40, 45, 50, 55, 60, 65 or 70 wt. % of a polyurethane polymer. It is an advantage that the amount of polymer to be used can be very low in comparison to other sustained release formulations.

The active agents that may be administered using the formulations, systems and methods of the invention are not limited, as the invention enables the effective delivery of a wide variety of active agents. The term “active agent/ingredient or drug” as used herein refers to therapeutic, diagnostic, cosmetic or prophylactic pharmaceutical and veterinary agents as well as other agents. The therapeutic or active agent may be selected from any of the various classes of such agents including, but not limited to, analgesic agents, anesthetic agents, anti-anginal agents, antiarthritic agents, anti-arrhythmic agents, antiasthmatic agents, antibacterial agents, anti-BPH agents, anticancer agents, anticholinergic agents, anticoagulants, anticonvulsants, antidepressants, antidiabetic agents, antidiarrheals, anti-epileptic agents, antifungal agents, antigout agents, antihelminthic agents, antihistamines, antihypertensive agents, antiinflammatory agents, antimalarial agents, antimigraine agents, antimuscarinic agents, antinauseants, antineoplastic agents, anti-obesity agents, antiosteoporosis agents, antiparkinsonism agents, antiprotozoal agents, antipruritics, antipsychotic agents, antipyretics, antispasmodics, antithyroid agents, antitubercular agents, antiulcer agents, anti-urinary incontinence agents, antiviral agents, anxiolytics, appetite suppressants, attention deficit disorder (ADD) and attention deficit hyperactivity disorder (ADHD) drugs, calcium channel blockers, cardiac inotropic agents, beta-blockers, central nervous system stimulants, cognition enhancers, corticosteroids, COX-2 inhibitors, decongestants, diuretics e.g. Hydrochlorothiazide (HCT), gastrointestinal agents, genetic materials, histamine receptor antagonists, hormonolytics, hypnotics, hypoglycemic agents, immunosuppressants, keratolytics, leukotriene inhibitors, lipid-regulating agents, macrolides, mitotic inhibitors, muscle relaxants, narcotic antagonists, neuroleptic agents, nicotine, nutritional oils, parasympatholytic agents, sedatives, sex hormones, sympathomimetic agents, tranquilizers, vasodilators, vitamins, and combinations thereof.

Active agents according to the invention also include nutrients, cosmeceuticals, diagnostic agents, and nutritional agents. Some agents, as will be appreciated by those of ordinary skill in the art, are encompassed by two or more of the aforementioned groups.

Anti-microbial agents such as broad spectrum antibiotics for combating clinical and sub-clinical infection, for example gentamycin, vancomycine and the like are also appropriate. Other suitable therapeutic agents are naturally occurring or synthetic organic or inorganic compounds well known in the art, including non-steroidal anti-inflammatory drugs, proteins and peptides (that may be produced either by isolation from natural sources or through recombination), hormones, bone repair promoters, carbohydrates, antineoplastic agents, antiangiogenic agents, vasoactive agents, anticoagulants, immunomodulators, cytotoxic agents, antiviral agents, antibodies, neurotransmitters, oligonucleotides, lipids, plasmids, DNA and the like.

The dosage form of the present invention can be particular useful for the oral delivery of (very) soluble drugs. Most matrices known in the art do not provide adequate control over the release rate of soluble or highly soluble drugs which often results in the problem of dose dumping or burst release. According to the European Pharmacopeia 2005, solubility can be determined as follows. In statements of solubility the terms used have the following significance referred to a temperature between 15° C. and 25° C.

| | Approximate volume of solvent in |
|---|---|
| Descriptive term | millilitres per gram of solute |
| Very soluble | less than 1 |
| Freely soluble | from 1 to 10 |
| Soluble | from 10 to 30 |
| Sparingly soluble | from 30 to 100 |
| Slightly soluble | from 100 to 1000 |
| Very slightly soluble | from 1000 to 10 000 |
| Practically insoluble | more than 10 000 |

As evident for a person skilled in the art, the load of the active agent(s) comprised in the dosage form according to this invention, may vary depending on the active agent(s) used and the envisaged application area. In general, the dosage form may comprise about and between 10-90 wt. % of active agent. In a specific embodiment the dosage form may comprise about and between 20-90 wt. % of active agent, or about and between 20-80 wt. % of active agent, and may comprise preferably at least or about 20%, more preferably at least or about 30 wt. %, 35 wt. %, 40 wt. %, 45 wt. %, 50 wt. %, 55 wt. %, 60 wt. %, 65 wt. %, 70 wt. %, or more wt. % of the active agent, including all values in between. As mentioned herein, any % is weight-by-weight, relative to the total weight of the dosage form or the formulation. As is evidenced by the present examples, the dosage form of the present invention is surprisingly useful for high drug loading, i.e. a drug loading of more than 35 wt. % can be obtained without the before mentioned disadvantages. In a particular embodiment, the invention encompasses a dosage form comprising at least 40 wt. % of an active agent, and even more particular at least 50 wt. % or 60 wt. % of an active agent.

In a further embodiment, the dosage form optionally comprises one or more components such as, but not limited to, a pore forming, a solubilizing and/or a wetting agent. A “pore forming” agent is well known to the skilled person and preferably is a water-soluble material such as sodium chloride, potassium chloride, sucrose, sorbitol, mannitol, polyethylene glycol (PEG), propylene glycol, hydroxypropyl cellulose, hydroxypropyl methycellulose, hydroxypropyl methycellulose phthalate, cellulose acetate phthalate, polyvinyl alcohols, methacrylic acid copolymers, poloxamers, including mixtures thereof. In a particular embodiment, the pore forming agent is PEG.

“Wetting agents and/or solubilizing agents” are well known to the skilled person and can be used to facilitate water ingress into the matrix and wetting of the drug in order to facilitate dissolution. Representative examples of wetting agents include gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethlylene castor oil derivatives, polyoxyethylene sorbitan Fatty acid esters (e.g., TWEENs™), polyethylene glycols (PEG), polyoxyethylene stearates. colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthlate, noncrystalline cellulose, magnesium aluminum silicate, benzalkoniumchloride, cetrimoniumbromide, natriumdioctylsulfosuccinaat, poloxamers, cylcodextrines, triethanolamine, polyvinyl alcohol, and polyvinylpyrrolidone (PVP). Preferred wetting agents include polyvinylpyrrolidone, polyethylene glycol, tyloxapol, and polyxamines, dextran, lecithin, and dialkylesters of sodium sulfosuccinic acid. Said components are commercially available. In a particular embodiment, the wetting agent is TWEEN.

The dosage form may comprise a single component of the ones mentioned hereinbefore or may comprise a combination of two or more of said components. In particular, the dosage form of the invention may comprise between 1-40 weight % of a pore forming, solubilizing and/or wetting agent, preferably about and between 1-5 wt. %, 1-10 wt. %, 1-15 wt. %, 1-20 wt. %, 1-25 wt. % or 1-30 wt. %, including all values in between, of a pore forming, solubilizing and/or wetting agent.

In a particular embodiment of this invention, the solid pharmaceutical dosage form as described herein provides a controlled release of the active agent wherein the majority i.e. about 50, 60, 70, 80, 85, 90, 95 or 100% of the active agent is gradually released within the first 96 hours after administration of the dosage form, preferably within the first 72, or 48 hours, most preferably within the first 24 hours. Preferably there is no burst off, i.e. such that after 1 hour not more than about 30% of the active agent is released. The release profile can be determined in vitro as described in the present examples, e.g. in USP hydrochloric acid (pH 1) at 37° C. in an USP Apparatus 2.

The extrusion process for preparing the oral release formulation of the current invention preferably is a melt extrusion process. The term “melt extrusion” relates to a process wherein an at least partially molten mass is formed and shaped. It includes, without being limited to, the process of hot melt extrusion and melt granulation.

The term “hot melt extrusion” is defined as a process by which material is mixed and at least partially melted then forced through a die under controlled conditions. “Melt granulation” is a process by which material is efficiently agglomerated and melted. The molten substance acts like a liquid binding and the dry granules are obtained as the molten binder solidifies by cooling. Dry agglomerates are obtained as the molten binding liquid solidifies by cooling.

In general in the melt extrusion process, the active agent is homogenized together with a polyurethane, and optionally a further component, followed by extrusion at about and between 60-200° C. Thereafter, the material is processed to the desired shape by moulding, injection moulding, rotation/injection moulding, casting, or other appropriate methods. The term “casting” refers to a process by which molten material is poured into a mold of a desired shape or onto a surface. The term “injection molding” relates to a process by which molten material is injected under pressure into a mold.

Typically, the extrusion process for preparing the oral dosage form according to this invention, can be described as follows. A composition comprising a polyurethane and an active agent is provided by mixing the aforementioned components supplied in the amounts as specified herein. Said components may be in the form of particles, but are preferably in powdered form and may optionally further be mixed with one or more additional components or excipients as disclosed herein. Although in some embodiments of the invention the composition to be mixed into the extruder may contain liquid materials, dry feed is advantageously employed in the extrusion process of the present invention. The mixtures are fed in an extruder and passed through a heated area of the extruder at a temperature which will melt or soften the compositions. Typical extrusion melt temperatures are from about 60° C. to about 200° C. More specific, the extrusion melt temperature is about 70° C.-160° C. The operating temperature range should be selected which will avoid the degradation or decomposition of the active agent and any other components of the composition during processing. The molten or softened mixtures then exit via a die or other element, at which time the mixtures (now called the extrudate) begin to harden. The extrudate can exit the extruder in various shapes, such as a film, sheet, rods, strands or other cross sections. Since the extrudate is still warm or hot upon exiting the extruder, it can be easily shaped or molded into various shapes, for example into a film, chopped, ground to powders, spheronized into beads or pellets, cut into strands, tableted or otherwise processed to the desired physical form by methods well known to the skilled person. For example, the extrudate can be processed to various solid pharmaceutical dosage forms by comminuting the extrudate in the shape of a film, sheet or strands into various forms, such as pellets, beads, granules or powders with known means, such as pelletizing, grinding or milling, and converting the particles to a dosage form.

More detailed methods for preparing to dosage form according to this invention are further provided in the examples hereinafter.

The extruded dosage forms are (inert) matrix formulations into which the drug is homogeneously embedded. The shape of the formulation or dosage form of the present invention can be cylindrical, angular, square, a flat sheet or any other shape, but preferably is cylindrical.

The pharmaceutical dosage form according to this invention may be in any suitable administration form. The dosage form is preferably formulated for oral or buccal drug delivery, and in particular, is for oral delivery for release of the active agent into the gastro-intestinal tract. Preferably, the release formulation of the present invention is shaped as or incorporated into solid dosage forms for oral administration such as but not limited to tablets, pills and capsules.

If desired, the compounds or the extruded composition of the present invention can be combined with pharmaceutical excipients to produce pharmaceutical dosage forms, such as one or more fillers, pigments, colorants, flavorants, disintegrating agents, binders, plasticizers, antioxidants, lubricants, solid diluents and/or liquid diluents. Examples of useful liquid diluents are oils, water, alcohols, or mixtures thereof, with or without the addition of pharmaceutically suitable surfactants, suspending agents, or emulsifying agents.

In a further aspect, the present invention provides an oral dosage form as defined herein for use as a medicament, in particular for the controlled release of an active ingredient. The dosage forms of the invention have a particular dissolution rate in vitro, said dosage forms providing an effective therapeutic effect for a sufficiently long period of time, in particular for at least 12 hours, more in particular for about 24 hours after oral administration. In particular the oral dosage forms of the invention are suited for dosing about every 24 hours.

The invention encompasses the use of the oral dosage form as described herein for the manufacture of a medicament for controlled release of the active agent. Furthermore, the present invention provides a method for the controlled release of an active agent, said method comprising orally administering to a patient a dosage form as defined herein.

The present invention also provides a method for preparing an oral dosage form according to this invention, said method comprising extruding a polyurethane and an active agent.

Finally, the present invention provides a dosage form obtainable by a process as defined herein.

This invention will be better understood by reference to the experimental details that follow, but those skilled in the art will readily appreciate that these are only illustrative of the invention as described more fully in the claims that follow thereafter. Particular embodiments and examples are not in any way intended to limit the scope of the invention as claimed. Additionally, throughout this application, various publications are cited. The disclosure of these publications is hereby incorporated by reference into this application to describe more fully the state of the art to which this invention pertains.

### EXAMPLES

### Example 1

Production of Injection Molded Tablets

Physical mixtures of drug/polymer at a 65/35 (wt. %) ratio were extruded at 70° C. using a co-rotating twin-screw extruder at 100 rpm (Haake MiniLab II Micro Compounder, Thermo Electron, Karslruhe, Germany). The polymer used was Pearlbond 539 (P539), the drugs used were Metoprolol tartrate (MPT), Theophylline (TH) and Diphylline (Dyph). Biconvex tablets (diameter: 10 mm/height: 5 mm) were produced via injection molding (Haake MiniJet System, Thermo Electron). The injection pressure was 800 bar during 10 s, in combination with a post-pressure of 400 bar for 5 s.

In Vitro Drug Release

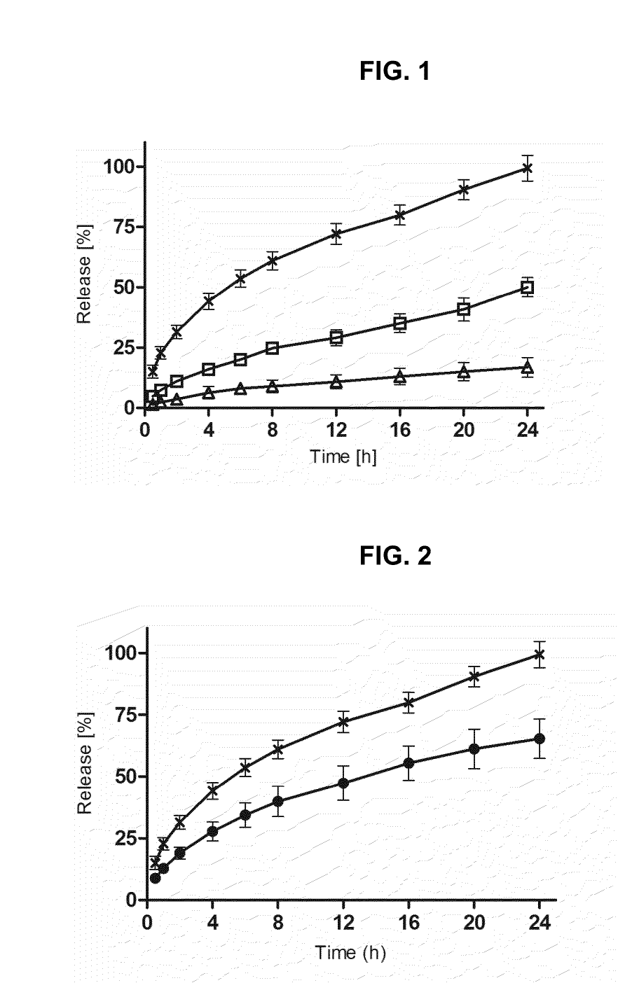

Drug release from the injection molded tablets was determined using the paddle method on a VK 7010 dissolution system (VanKel Industies, N.J., USA) with a paddle speed of 100 rpm. Distilled water was used as dissolution medium (900 mL) at 37±0.5° C. Samples were withdrawn at 0.5, 1, 2, 4, 6, 8, 12, 16, 20 and 24 h and spectrophotometrically analyzed for API concentration at 272 nm for Th and 274 nm for MPT and Dyph, respectively (FIG. 1).

The paddle apparatus (Apparatus II) consists of a special, coated paddle that minimizes turbulence due to stirring. The paddle is attached vertically to a variable-speed motor that rotates at a controlled speed. The position and alignment of the paddle are specified in the USP (Chapter <711>).

### Example 2

Production of Injection Molded Tablets

Physical mixtures of drug/polymer at ratio of 50/50, 60/40 and 65/35 (wt. %) were extruded at 70° C. using a co-rotating twin-screw extruder at 100 rpm (Haake MiniLab II Micro Compounder, Thermo Electron, Karslruhe, Germany). The polymer used was Pearlbond 523 (P523), the drug Metoprolol tartrate (MPT). Biconvex tablets (diameter: 10 mm/height: 5 mm) were produced via injection molding (Haake MiniJet System, Thermo Electron). The injection pressure was 800 bar during 10 s, in combination with a post-pressure of 400 bar for 5 s.

In Vitro Drug Release

Drug release from the injection molded tablets was determined using the paddle method on a VK 7010 dissolution system (VanKel Industies, N.J., USA) with a paddle speed of 100 rpm. Distilled water was used as dissolution medium (900 mL) at 37±0.5° C. Samples were withdrawn at 0.5, 1, 2, 4, 6, 8, 12, 16, 20 and 24 h and spectrophotometrically analyzed for MPT concentration at 274 nm (FIG. 2).

### Example 3

Production of Injection Molded Tablets

Physical mixtures of drug/polymer at ratio of 50/50 and 65/35 (wt. %) were extruded at 70° C. using a co-rotating twin-screw extruder at 100 rpm (Haake MiniLab II Micro Compounder, Thermo Electron, Karslruhe, Germany). The polymer used was Pearlbond 539 (P539), the drug Dyphilline (Dyph). To further optimize drug release, a pore former, PEG4000 was added to the Dyph/P539 mixture at 2, 5 and 10%, respectively. Biconvex tablets (diameter: 10 mm/height: 5 mm) were produced via injection molding (Haake MiniJet System, Thermo Electron). The injection pressure was 800 bar during 10 s, in combination with a post-pressure of 400 bar for 5 s.

In Vitro Drug Release

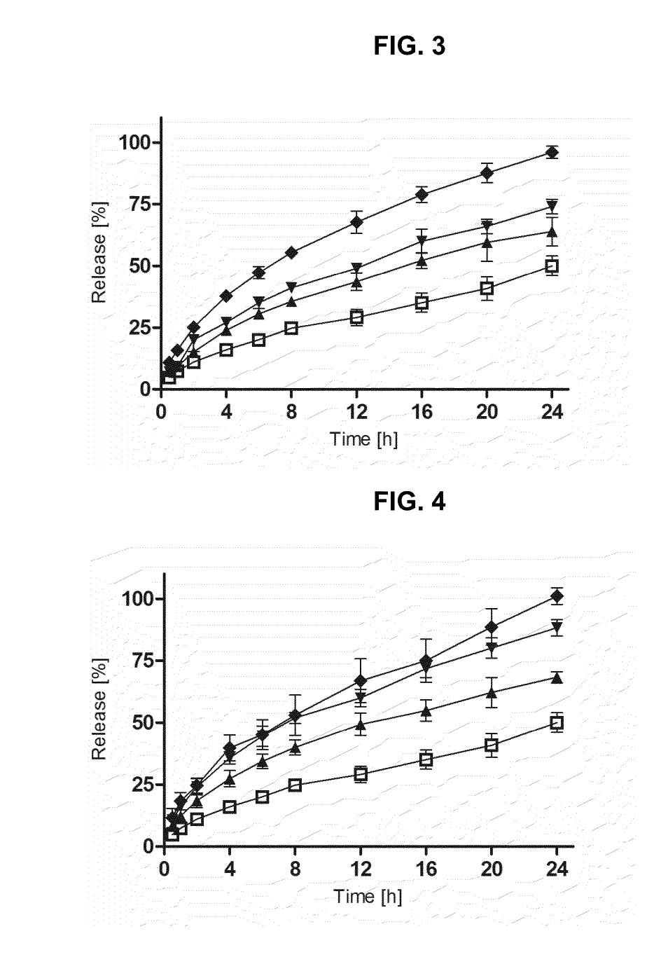

Drug release from the injection molded tablets was determined using the paddle method on a VK 7010 dissolution system (VanKel Industies, N.J., USA) with a paddle speed of 100 rpm. Distilled water was used as dissolution medium (900 mL) at 37±0.5° C. Samples were withdrawn at 0.5, 1, 2, 4, 6, 8, 12, 16, 20 and 24 h and spectrophotometrically analyzed for Dyph concentration at 274 nm (FIG. 3).

### Example 4

Production of Injection Molded Tablets

Physical mixtures of drug/polymer at ratio of 50/50 and 65/35 (wt. %) were extruded at 70° C. using a co-rotating twin-screw extruder at 100 rpm (Haake MiniLab II Micro Compounder, Thermo Electron, Karslruhe, Germany). The polymer used was Pearlbond 539 (P539), the drug Dyphilline (Dyph). To alter drug release, a pore former, Tween80 was added to the Dyph/P539 mixture at 2, 5 and 10%, respectively. Biconvex tablets (diameter: 10 mm/height: 5 mm) were produced via injection molding (Haake MiniJet System, Thermo Electron). The injection pressure was 800 bar during 10 s, in combination with a post-pressure of 400 bar for 5 s.

In Vitro Drug Release

Drug release from the injection molded tablets was determined using the paddle method on a VK 7010 dissolution system (VanKel Industies, N.J., USA) with a paddle speed of 100 rpm. Distilled water was used as dissolution medium (900 mL) at 37±0.5° C. Samples were withdrawn at 0.5, 1, 2, 4, 6, 8, 12, 16, 20 and 24 h and spectrophotometrically analyzed for Dyph concentration at 274 nm (FIG. 4).

### Example 5

Production of Injection Molded Tablets

Physical mixtures of drug/polymer at ratio 65/35 (wt. %) with 7.5 and 10% PEG4000, respectively, were extruded at 140° C. using a co-rotating twin-screw extruder at 100 rpm (Haake MiniLab II Micro Compounder, Thermo Electron, Karslruhe, Germany). The polymer used was Tecoflex80A (T80A), the drug Dyphilline (Dyph). Biconvex tablets (diameter: 10 mm/height: 5 mm) were produced via injection molding (Haake MiniJet System, Thermo Electron). The injection pressure was 800 bar during 10 s, in combination with a post-pressure of 400 bar for 5 s.

In Vitro Drug Release

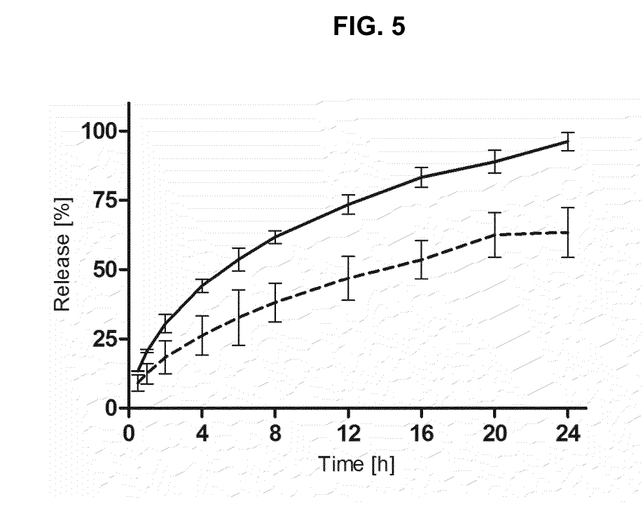

Drug release from the injection molded tablets was determined using the paddle method on a VK 7010 dissolution system (VanKel Industies, N.J., USA) with a paddle speed of 100 rpm. Distilled water was used as dissolution medium (900 mL) at 37±0.5° C. Samples were withdrawn at 0.5, 1, 2, 4, 6, 8, 12, 16, 20 and 24 h and spectrophotometrically analyzed for Dyph concentration at 274 nm (FIG. 5).

### Example 6

Production of Injection Molded Tablets

Physical mixtures of drug/polymer at ratio of 65/35 (wt. %) were extruded at 70° C. using a co-rotating twin-screw extruder at 100 rpm (Haake MiniLab II Micro Compounder, Thermo Electron, Karslruhe, Germany). The polymer used was Pearlbond 539 (P539), the drug Metoprolol tartrate (MPT). Biconvex tablets (diameter: 10 mm/height: 5 mm) were produced via injection molding (Haake MiniJet System, Thermo Electron). The injection pressure was 800 bar during 10 s, in combination with a post-pressure of 400 bar for 5 s.

In Vivo Drug Release

All procedures were performed in accordance with the guidelines and after approval by the Ethics Committee of the Institute for Agricultural and Fisheries Research (ILVO) (Merelbeke, Belgium). To study the influence of MPT concentration, the following formulations were administrated to dogs:
Formulation 1 (F1): IM tablets containing 65% MPT and 35% Pearlbond 539 (administrated to 3 dogs);
Formulation 2 (F2): Slow-Lopresor® 200 Divitabs® (Sankyo, Louvain-la-Neuve, Belgium), a commercial sustained release formulation consisting of matrix tablets containing 200 mg MPT (administrated to 6 dogs).

Slow-Lopresor® 200 Divitabs® (1 tablet) was used as a reference formulation (F2). All formulations were administrated to male mixed-breed dogs (10-13 kg) in a cross-over study with a wash-out period of at least 8 days. The dogs were fasted 12 h prior to the administration and 12 h after administration, although water was available ad libitum. Before the administration, an intravenous cannula was placed in the lateral saphenous and a blank blood sample was collected. The formulations were administrated with 20 ml water and blood samples were collected in dry heparinized tubes at 0.5, 1, 2, 4, 6, 8, 12, 16, 20 and 24 h after administration. The obtained blood samples were centrifugated at 1500 g during 5 min.

Metoprolol Tartrate Assay

A validated HPLC method with fluorescence detection was used for the determination of MPT in dog plasma. Plasma samples (300 μl) were mixed with 20 μl of a 2.75 μg/ml aqueous bisoprolol hemifumarate (internal standard, IS) solution and 320 μl of a 4% (v/v) aqueous phosphoric acid solution. The mixtures were vortexed during 30 s. Drug and internal standard were extracted using solid phase extraction (SPE) cartridges (Oasis® MCX 1 cc (30 mg), Waters, Brussels, Belgium) and a 10-port vacuum extraction manifold. After conditioning the SPE columns with 1 ml methanol and 1 ml water, the plasma samples were transferred to the columns. The columns were rinsed with 1 ml of a 2% (v/v) aqueous formic acid solution, followed by 1 ml methanol. 1 ml of a 5% (v/v) solution of ammonium hydroxide in methanol was used to elute MPT and IS. Samples were evaporated to dryness under a N2-flow and reconstituted in 150 μl distilled water. 20 μl was injected into the HPLC system. MPT plasma concentrations were calculated from a calibration curve, determined by adding 20 μl of a MPT standard (0.375, 0.5625, 0.75, 1.5, 2.25, 3.75 and 5.25 μg/ml), 20 μl of the IS solution and 320 μl of an aqueous phosphoric acid solution (4% (v/v)) to 280 μl of blank plasma. The mixtures were then treated as described previously. The method validation indicated a linear relationship between MPT plasma concentration and response (range: 0-353.5 ng/ml; R2=0.999±0.001 (n=8)). The limit of detection and limit of quantification were 10.1 and 30.6 ng/ml, respectively. MPT showed a retention time of 14 min, while the IS eluted after 18 min. The HPLC system consisted of an isocratic solvent pump (L-7100, Merck, Hitachi LaChrom, Tokyo, Japan), an automatic autosampler (L-2200, Merck, Elite LaChrom, Tokyo, Japan), a guard column (LiChroCart® 4-4, LiChrospher® 100 CN (5 μm), Merck, Darmstadt, Germany) followed by a reversed phase CN column (LiChroCart® 250-4, LiChrospher® 100 CN (5 μm), Merck, Darmstadt, Germany) and a variable wavelength fluorescence detector (L-7480, Merck, Hitachi LaChrom, Tokyo, Japan). Peak integration was performed using the software package D-7000 HSM Chromatography Data Station (Hitachi Instruments, San 11 Jose, Calif., USA). The mobile phase consisted of a phosphate buffer solution (2M sodium phosphate monobasic dihydrate), acetonitrile and water (0.5/3.5/96; v/v/v) adjusted to pH 3 with phosphoric acid. The pump flow was set at 1.1 ml/min and the excitation and emission wavelength were 275 nm and 300 nm, respectively.

Data Analysis

The peak plasma concentration (Cmax) and the time needed to reach the highest plasma concentration (tmax) were determined. The controlled release characteristics of the formulations were evaluated by means of the HVDt50% Cmax (halfvalue duration) defined by the period during which the plasma concentration exceeds 50% of the Cmax. The effect of the formulation on the bioavailability was statistically evaluated by repeated-measures ANOVA (univariate analysis) using SPSS 17 (SPSS, Chicago, USA). To compare the effects of the different treatments on the pharmaco-kinetic parameters, a multiple comparison among pairs of means was performed using a Bonferroni post-hoc test with p<0.05 as significance level.

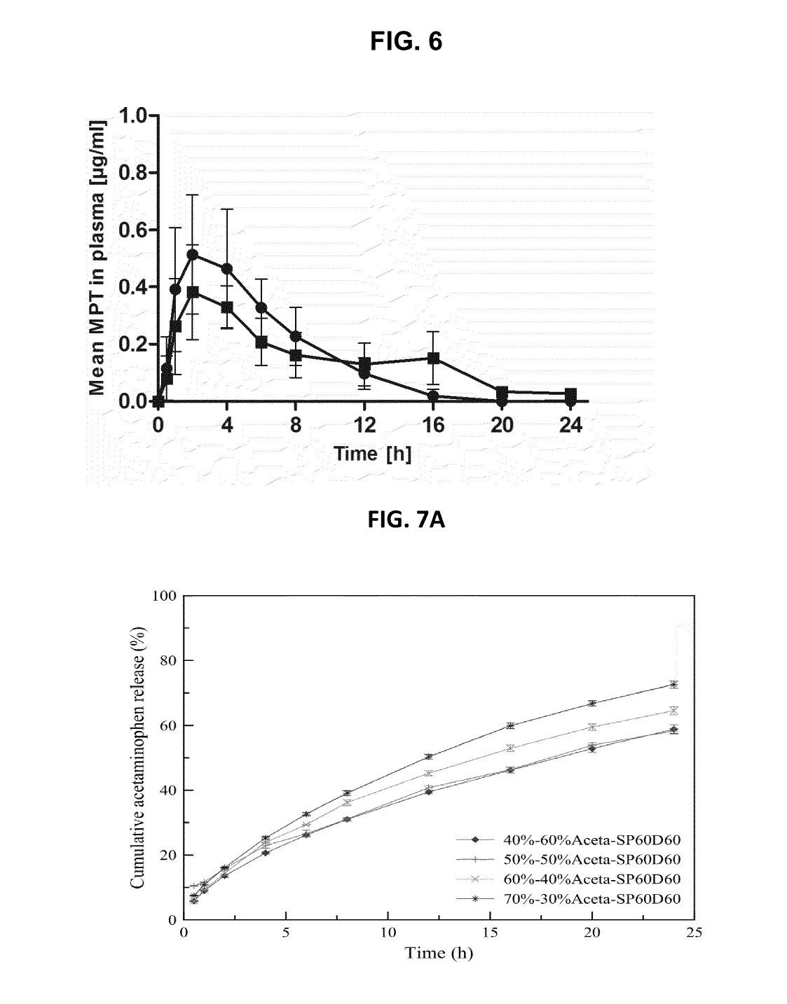

As shown in FIG. 6 no significant differences were observed between the release characteristics of the formulations F1 and F2.

### Example 7

Production of HME/IM Tablets in Combination with Different Model Drugs

Various grades of hydrophilic Tecophilic™ TPUs were obtained from Merquinsa (a Lubrizol Company, Ohio, USA). The hard segment of these hydrophilic and aliphatic TPUs is hexamethylene diisocyanate (HMDI) in combination with 1,4-butanediol (1,4-BD) as chain extender, while its soft segment is poly (ethylene oxide) (PEO). Different Tecophilic™ grades were evaluated: aliphatic extrusion-grade TPUs (HP60D20, HP60D35, HP60D60 and HP93A100), solution-processable TPUs (SP60D60, SP93A100 and SP80A150) and hydrogel TPUs (TG500 and TG2000).

Theophylline (Theo), diprophylline (Dyph, 7-(2,3-dihydroxypropyl)-theophylline) and acetaminophen (Aceta) (Sigma Aldrich, Bornem, Belgium) were used as model drugs to investigate whether Tecophilic™ grades allowed to sustain release of highly dosed drugs with different aqueous solubility without using release modifiers.

HME/IM was performed on selected TPUs in combination with diprophylline, acetaminophen and theophylline (aqueous solubility in 100 ml at 25° C.: 33, 1.4 and 0.7 g, respectively). Physical mixtures (50% drug load, w/w, in all cases) were extruded using a co-rotating twin-screw extruder (Haake MiniLab II Micro Compounder, Thermo Electron, Karlsruhe, Germany), operating at different screw speeds (50, 75 and 100 rpm) and processing temperatures (110, 120, 130 and 140° C. for SP60D60 formulations; 110° C. for SP93A100 formulations and 80° C. for TG2000 formulations). To evaluate the effect of drug load, physical mixtures of drug (concentration was varied from 40-80% (w/w)) and Tecophilic™ SP60D60 were processed via HME at 100 rpm using a barrel temperature of 110° C.

After HME, the extrudates were immediately processed into tablets via Injection Moulding (Haake MiniJet System, Thermo Electron, Karlsruhe, Germany) at a temperature equal to the extrusion temperature. During the IM process an injection pressure of 800 bar (during 10 s) forces the material into the mould. A post-pressure of 400 bar (during 5 s) avoids expansion by relaxation of the polymer. As not only HME processing parameters might affect drug release, injection moulding pressure and post-injection pressure were varied from 600-1000 bar and 200-600 bar, respectively.

In Vitro Dissolution

Drug release from the injection-moulded tablets was determined using the paddle method on a VK 7010 dissolution system (VanKel Industries, New Jersey, USA) with a speed of 100 rpm. Distilled water is used as dissolution medium (900 mL) at 37±0.5° C. Samples were withdrawn at predetermined time points (0.5; 1; 2; 4; 6; 8; 12; 16; 20 and 24 h) and spectrophotometrically (UV-1650PC, Shimadzu Benelux, Antwerp, Belgium) analysed. In vitro drug release data were fitted to zero order release kinetics and R2 values were calculated. At the same time points, formulations are withdrawn from the medium and weighed after removing excess surface water. Images are made with a digital camera (C3030 Olympus) attached to an image analysis system (analySISC) to visualize swelling behaviour. A digital calliper (Bodson, Luik, Belgium) is used to measure the height and diameter of the injection-moulded tablets in order to determine the axial and radial swelling, respectively. The water uptake (% weight gain of the hydrophilic TPU) is calculated from the weight of the mini-matrices, taking into account the drug released as described in the following equation.

$$\% water uptake = \frac{( Ww - Drt ) - ( Wi - Dr 0 )}{( Wi - Dr 0 )} \times 100$$

Where Ww=weight of the mini-matrix at time ‘t’ (hours after immersion)

Wi=initial weight of the mini-matrix before dissolution

Dr0=amount of drug in the mini-matrix before dissolution

Drt=amount of drug in the mini-matrix at time ‘t’ (hours after immersion)

The effect of HME/IM process temperature, extrusion speed, drug load and injection pressure on in vitro drug release and swelling behaviour is determined to evaluate robustness.

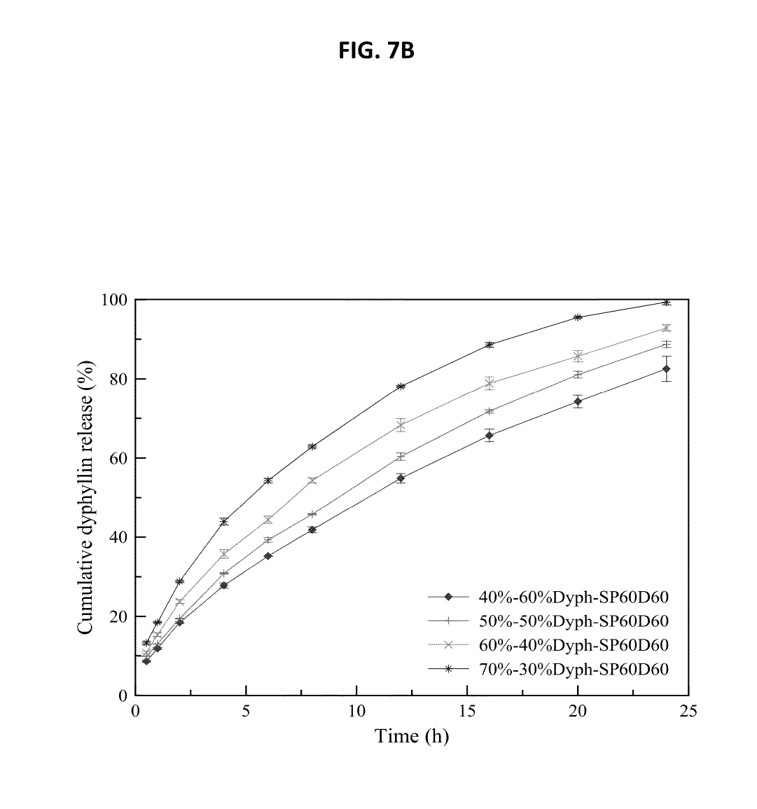

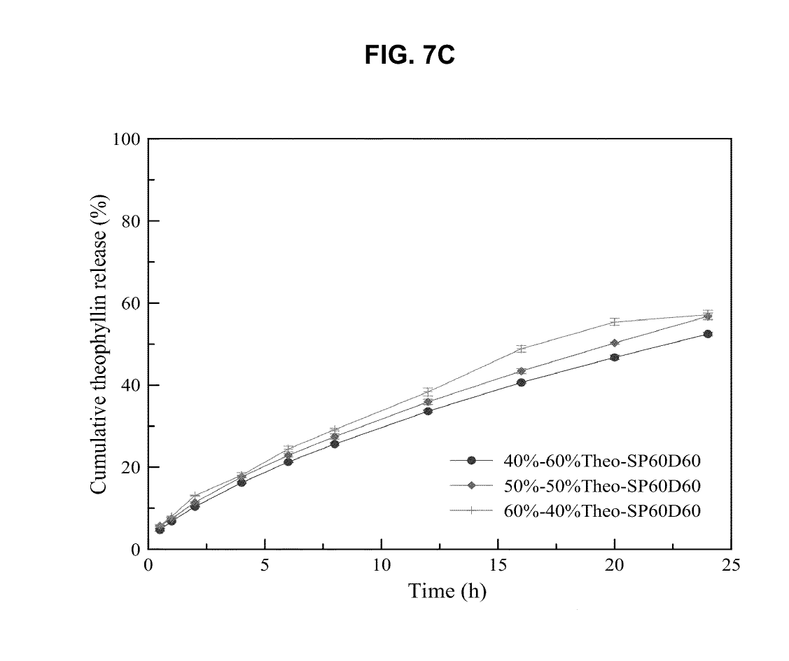

Besides the length of the soft segment, drug release was affected by drug loading as TPU matrices with a high drug load (up to 70%, w/w) yielded faster release kinetics (FIGS. 7A-C). Similar to the previous results no burst-effect was observed. In addition, release kinetics of all model drugs were not affected by modifying HME screw speed, barrel temperature nor by changing downstream processing parameters (i.e. injection pressure and post-injection pressure).

### Example 8

Production of Metformin.HCL Polyurethane Matrices

Both, the hydrophobic TPU grade Tecoflex™ EG72D and the hydrophilic TPU grades Tecophilic™ SP60D60, SP93A100 and TG2000 were obtained from Merquinsa (a Lubrizol Company, Ohio, USA). Metformin.HCl was purchased from Fagron (Belgium) and was embedded as model drug to investigate whether a mixture of Tecoflex™ and Tecophilic™ grades allows adjustment of the release kinetics.

Production of HME/IM Tablets and Multiparticulate Dosage Forms

HME is performed on the mentioned TPUs in combination with metformin.HCl (60% drug load in all cases). Physical mixtures were extruded using a co-rotating twin-screw extruder (Haake MiniLab II Micro Compounder, Thermo Electron, Karlsruhe, Germany), operating at a screw speed of 100 rpm and processing temperature of 100° C. for formulations containing TG2000 and 160° C. for formulations containing other hydrophilic TPUs and/or the hydrophobic TPU Tecoflex™ EG72D.

After HME, the extrudates were immediately processed into tablets via Injection Moulding or calendaring. Injection Moulding was performed using a Haake MiniJet System (Thermo Electron, Karlsruhe, Germany) at a temperature equal to the extrusion temperature. During the IM process an injection pressure of 800 bar (10 s) forces the material into the mould. A post-pressure of 400 bar (5 s) avoids expansion by relaxation of the polymer. Mini-matrices (±3.5 mm length; ±3 mm diameter) were obtained by calendaring the extrudates with a surgical blade.

In Vitro Release

The in vitro release experiments are based on the USP guidelines of metformin hydrochloride sustained release tablets which include the employment of an USP standard apparatus II. Drug release from the injection-moulded tablets and mini-matrices was determined using the paddle method on a VK 7010 dissolution system (VanKel Industries, New Jersey, USA) with a speed of 100 rpm. SIF, SGF and SGF+ethanol (20/80 and 40/60% (V/V) ethanol/SGF, respectively) were used as dissolution media (900 mL) at 37+/−0.5° C. Samples were withdrawn at predetermined time points (0.5; 1; 2; 4; 6; 8; 12; 16; 20 and 24 h) and spectrophotometrically (UV-1650PC, Shimadzu Benelux, Antwerp, Belgium) analysed at a wavelength of 232 nm.

Images were made with a digital camera (C3030 Olympus) attached to an image analysis system (analySIS®) to visualize swelling behaviour. A digital calliper (Bodson, Luik, Belgium) is used to measure the height and diameter of the injection-moulded tablets in order to determine the axial and radial swelling, respectively.

In Vivo Release

An ethical application was submitted and approved by the Ethics comity of veterinary medicine (University of Ghent) before starting the experiments.

Subjects and Study Design

In vivo experiments were performed using the following formulations: mini-matrices (60/40, w/w (%), metformin.HCl/Tecoflex™ EG 72D) and IM tablets (60/20/20, w/w (%), metformin.HCl/SP60D60/EG 72D)). Both formulations were compared with Glucophage® SR 500 mg (. tablet) as a reference. Open label cross-over assays were performed on 6 male beagle dogs (10-13 kg) with a wash-out period of at least 8 days. The IM tablets, mini-matrices and reference formulation were administrated sober with 20 mL of water. During the experiment the dogs were only allowed to drink water. Blood samples were collected after 1, 2, 3, 4, 5, 6, 8 and 12 hours post oral administration and were stored at −25° C. until extraction. All TPU based formulations were recovered from faeces and residual metformin.HCl content was immediately determined after 24 h dissolution experiments (USP standard apparatus II dissolution bath, paddle stirrer at 100 rpm, phosphate buffer (pH 6.8) dissolution medium at 37° C. and sample collection after 24 h).

Metformin.HCl Assay

An extraction method developed by Gabr et al. was optimized and used. After de-freezing, blood serum samples were centrifuged using a Centric 322A (Tehtnica, Slovenia) at 3000 rpm for 10 minutes. 280 μL of the supernatant was spiked with 20 μL of 0.05 mg/mL ranitidine solution. During a first extraction step, 50 μL of 10 M sodium hydroxide solution and 3 mL organic phase (1-butanol/hexane, 50/50, V/V) were added. The mixture was vortexed and mixed during 30 seconds and 30 minutes, respectively. The upper organic layer was transferred to a clean test tube after centrifugation. Back extraction was performed by adding 1 mL of 2M HCl. Consecutively, the mixture was vortexed, mixed and centrifuged. After centrifugation (10 min, 3000 rpm) the organic layer was removed, 400 μL of sodium hydroxide (10 M) and 2 mL organic phase (1-butanol/hexane, 50/50, V/V) were added. After vortexing, mixing and centrifugation, the organic layer was transferred into a clean glass tube and evaporated to dryness under a nitrogen stream.

The HPLC system (Merck-Hitachi, Darmstadt, Germany) consisted of an isocratic solvent pump (L-7100) set at a constant flow rate of 0.7 mL/min, an auto-sampler injection system (L-7200) with a 100 μL loop (Valco Instruments Corporation, Houston, Tex., USA), a reversed-phase column and precolumn (LiChroCartR 250-4 and LiChrospherR 100RP-18 5 μm, respectively) and a variable wavelength UV-detector (L-7400) set at 236 nm. The mobile phase consisted of potassium dihydrogen phosphate buffer (adjusted to pH 6.5 with 2M NaOH)/acetonitrile (66/34, V/V) and 3 mM sodium dodecyl sulphate (SDS). Peak integration was performed using the software package D-7000 HSM Chromatography Data Station.

The potential of the flexible TPU based matrix system was confirmed in vivo.

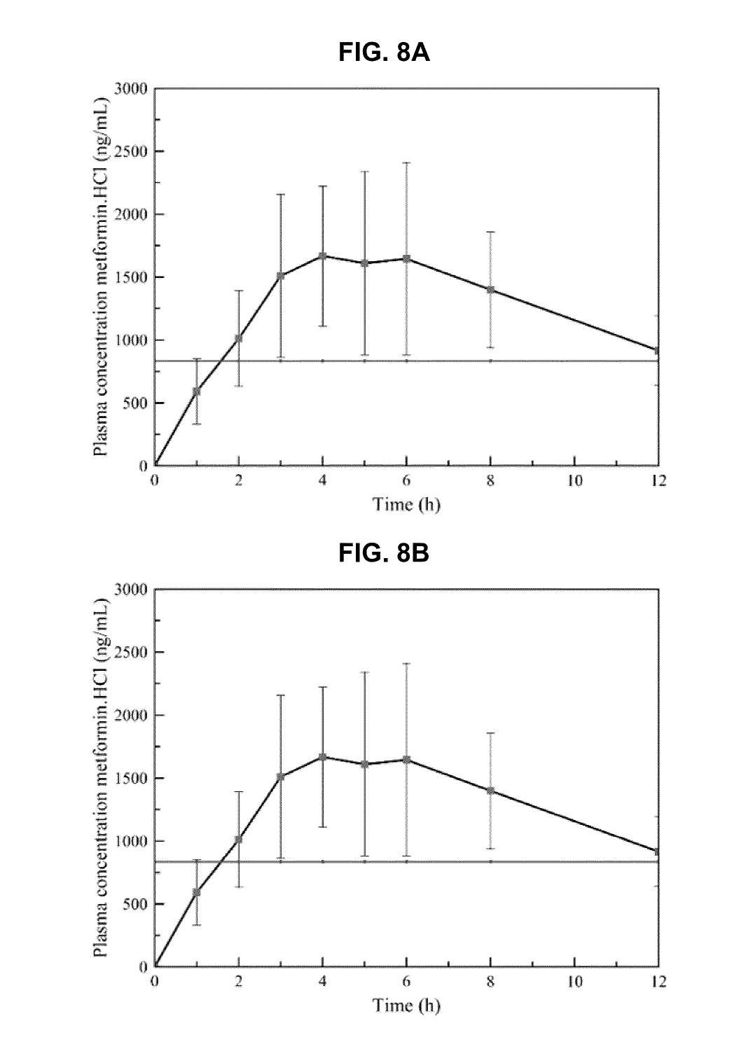

The blood level of the anti-diabetic Metformin-HCl after administration of injection-molded TPU tablets was sustained during 10.43 h (HVDT50% Cmax), vs. 6.25 h for Glucophage® SR, which was used as a reference. The corresponding RD-value of the TPU tablet was 3.26, indicating strong sustained release, whereas Glucophage® SR showed an intermediate sustained release profile (RD=1.96) (FIGS. 8A and 8B).

### REFERENCES

Batyrbekov Y. and Iskakov R., 2012, “Polyurethane” Ed. Zafar and Sharmin—Chapter 8: Polyurethane as Carriers of antituberculosis drugs, p 147-170.

Cherng J Y et al., 2013, International Journal of Pharmaceutics 450: 145-162.

Clark et al., 2012, Journal of Pharmaceutical Sciences 101(2): 576-587.

K. Hong, S. Park, 1999, Reactive and Functional Polymers, 42: 193-200.

Subhaga C S et al., 1995, J Microencapsulation 12(6): 617-625.

Gabr, R. Q., Padwal, R. S., Brocks, D. R., (2010) Determination of metformin in human plasma and urine by high-performance liquid chromatography using small sample volume and conventional octadecyl silane column, J Pharm Pharm Sci, 13, 486-494.

What is claimed is:

1. A controlled release oral dosage form, wherein said dosage form comprises an extrudate comprising:
a polyurethane, and
at least 40 weight % of an active agent.

2. The dosage form according to claim 1, wherein the polyurethane is a thermoplastic polyurethane.

3. The dosage form according to claim 1, wherein the polyurethane is present at an amount of between and about 10-60 weight % of the dosage form.

4. The dosage form according to claim 1, further comprising at least one pore forming, solubilizing and/or wetting agent.

5. The dosage form according to claim 1, wherein said dosage form is in the shape of a tablet, pill or capsule.

6. The dosage form according to claim 1, wherein the dosage form is a single-layered release formulation, or wherein the dosage form does not comprise a drug impermeable coating.

7. The dosage form according to claim 1, wherein the active agent is a very soluble to soluble drug as determined according to the definition in the European Pharmacopeia 2005.

8. The dosage form according to claim 1, wherein the oral dosage form is in a suitable form for the release of the agent in the gastro-intestinal tract.

9. A pharmaceutical composition comprising an oral dosage form according to claim 1, and a pharmaceutically acceptable excipient.

10. A method of preparing an oral dosage form, said method comprising:
providing a polyurethane and an active agent, and
melt extruding said components or a mixture thereof,
characterized in that the dosage form comprises at least 40 wt. % of the active agent.

11. The method according to claim 10, wherein the melt extrusion is hot melt extrusion or melt granulation.

12. The method according to claim 10, further comprising injection molding or casting.

13. An oral dosage form obtainable by a method according to claim 10.

14. A method for the controlled release of an active agent, said method comprising orally administering to a subject the dosage form as defined in claim 1.

15. Method according to claim 14, wherein the controlled release is characterised by a gradual release of at least 90% of the active agent within 24 hours.
